(19) United States
(12) Patent Application Publication
(10) Pub. No.: US 2019/0366080 A1
(43) Pub. Date: Dec. 5, 2019

(54) SUBCUTANEOUS TUNNELING TOOL WITH GUIDING MECHANISMS

(71) Applicant: Florida Electrophysiology LLC, Orlando, FL (US)

(72) Inventor: Usman Siddiqui, Orlando, FL (US)

(21) Appl. No.: 15/991,878

(22) Filed: May 29, 2018

(51) Int. Cl. A61N 1/05; A61N 1/39

(52) U.S. Cl. CPC A61N 1/0563; A61B 2017/320056; A61N 1/3968

(57) ABSTRACT

A subcutaneous tunneling tool comprises an elongated shaft that may be inserted through an incision in a patient to create a subcutaneous tunnel under the patient's skin. The subcutaneous tunneling tool also comprises a guide mechanism secured to a proximal end of the elongated shaft, wherein the guide mechanism is configured to project a light beam aligned with the elongated shaft toward the elongated shaft. When at least a distal end of the elongated shaft is positioned beneath a patient's skin, the light beam is projected onto an external surface of the patient's skin, aligned with the elongated shaft such that the light beam may be used to guide the elongated shaft along a desired tunnel path.

### BACKGROUND

Implantable cardiac rhythm management systems, such as Implantable cardioverter/defibrillators (ICDs), pace makers, and/or the like, have been used for many years to treat patients with serious cardiac arrhythmias. These systems, which generally include a number of electrical leads and sensors extending away from a controller module, are generally implanted subcutaneously to protect the device itself and to increase patient comfort. The electrical leads of these devices are generally routed into the interior of the patient's heart (often through portions of the patient's arteries) such that the device is capable of applying high-intensity electrical pulses for treating various arrhythmias. The associated sensors are also secured to the patient's heart, typically at various interior and exterior surfaces such that those sensors may accurately detect the heart's electrical signals to ensure that electrical pulses from the ICDs are provided effectively to treat and/or counteract the patient's arrhythmia.

Controller modules of the ICDs (which may include power supplies, control circuitry, and/or the like) are generally implanted a distance away from the patient's heart, such as between the fifth and sixth intercostal spaces of the patient's rib cage. The attached electrical leads and sensors are then routed along a minimally invasive thread path from the controller to the patient's heart.

Although the thread path for the various electrical leads and sensors is selected to minimize the amount of tissue damage experienced by the patient, there is a continuing need for systems and methods for further reducing the amount of tissue damage experienced by the patient during ICD implant procedures to minimize the amount of recovery time needed by the patient post-surgery.

### BRIEF SUMMARY

A subcutaneous tunneling tool comprising one or more integrated guide mechanisms is provided to aid cardiac surgeons in implanting ICDs and their associated electrical leads and sensors in a patient. The tunneling tool itself creates a subcutaneous thread path used to route the electrical leads and sensors within the patient's body. The integrated guide mechanisms ensure that the surgeon is creating a subcutaneous thread path that is minimally invasive, for example by creating at least substantially linear portions of a thread path having a desired constant subcutaneous depth.

Various embodiments are directed to a subcutaneous tunneling tool comprising: an elongated shaft configured to be inserted below a patient's skin to create a subcutaneous tunnel; and a housing comprising a guide mechanism secured to an end of the elongated shaft, wherein the guide mechanism is configured to project a visible light beam aligned with the elongated shaft toward the elongated shaft.

In certain embodiments, the guide mechanism comprises a beam generator secured to the housing and spaced apart from the elongated shaft, wherein the beam generator is configured to project the visible light beam toward the elongated shaft. In certain embodiments, the guide mechanism further comprises a leveling indicator secured to the housing. Moreover, the beam generator is configured to project a laser line toward the elongated shaft, wherein the laser line is aligned with the elongated shaft. In certain embodiments, the laser line is longer than the elongated shaft. Moreover, the elongated shaft may comprise a metallic material. Moreover, the subcutaneous tunneling tool may further comprise an elongated sheath surrounding the elongated shaft. In certain embodiments, the elongated sheath has length demarcations thereon. Moreover, the subcutaneous tunneling tool may further comprise a vibration emitter configured to vibrate the elongated shaft at a defined frequency to separate subcutaneous tissue of the patient when the elongated shaft is inserted below the patient's skin. Moreover, the defined frequency may be a radio frequency.

Various embodiments are directed to a method for creating a subcutaneous tunnel in a patient. In various embodiments, the method comprises: inserting a distal end of an elongated shaft through an incision in the patient's skin; projecting a guide beam onto an exterior surface the patient's skin, wherein the guide beam is projected from a guide mechanism secured to a proximal end of the elongated shaft and the guide beam is aligned with the elongated shaft; aligning the guide beam with a desired subcutaneous tunnel path; and inserting the elongated shaft through the desired subcutaneous tunnel path.

In various embodiments, the method further comprises vibrating the elongated shaft at a desired frequency to separate subcutaneous tissue along the desired subcutaneous tunnel path. Moreover, the desired frequency may be a radio frequency. In certain embodiments, the method further comprises steps for aligning a leveling indicator within the guide mechanism with a level configuration and maintaining the leveling indicator in the level configuration while inserting the elongated shaft through the desired subcutaneous tunnel path. In certain embodiments, the elongated shaft is surrounded by a sheath having length demarcations thereon, and wherein inserting the elongated shaft through the desired subcutaneous tunnel path comprises inserting the elongated shaft through the incision to a desired length demarcation. Moreover, the distal end of the elongated shaft may be exposed beyond a distal end of the sheath, wherein the method may further comprise vibrating the elongated shaft at a desired frequency within the sheath to separate subcutaneous tissue at the distal end of the elongated shaft.

### BRIEF DESCRIPTION OF THE SEVERAL VIEWS OF THE DRAWINGS

Reference will now be made to the accompanying drawings, which are not necessarily drawn to scale, and wherein:

FIG. 1 is a perspective view of a subcutaneous tunneling tool according to one embodiment;

FIG. 2 is another perspective view of the subcutaneous tunneling tool shown in FIG. 1;

FIG. 3 is an exploded view of the subcutaneous tunneling tool shown in FIG. 3; and

FIG. 4 is a perspective view of a subcutaneous tunneling tool according to one embodiment as it is inserted through an incision in a patient's skin.

### DETAILED DESCRIPTION

The present disclosure more fully describes various embodiments with reference to the accompanying drawings. It should be understood that some, but not all embodiments are shown and described herein. Indeed, the embodiments may take many different forms, and accordingly this disclosure should not be construed as limited to the embodiments set forth herein. Rather, these embodiments are provided so that this disclosure will satisfy applicable legal requirements. Like numbers refer to like elements throughout.

Overview

A subcutaneous tunneling tool according to various embodiments comprises a body portion usable as a handle for a user (e.g., a surgeon) and housing a laser guide mechanism. The subcutaneous tunneling tool further comprises a shaft extending away from the handle. The shaft is configured to be inserted through an incision in the patient's skin to form the subcutaneous thread path. The shaft may be a conductive (e.g., metallic) material, and may comprise a non-conductive sleeve having length demarcations along the length of the sleeve.

The laser guide mechanism of the housing is configured to project a visible laser line that is aligned with the length of the shaft. The laser guide mechanism is positioned within the housing such that, when the shaft is at least partially inserted into a patient, the laser guide mechanism projects the laser onto the exterior surface of the patient's skin, such that the user can align the projected laser (and correspondingly the shaft) with a desired thread path. Moreover, the body portion may further comprise a leveling indicator (e.g., a bubble level) that may be used to ensure the shaft is inserted at a desired, consistent depth relative to the patient's skin.

In certain embodiments, the subcutaneous tunneling tool may be configured to emit a vibration frequency along the length of the shaft (e.g., at a radio frequency level) to aid in insertion of the shaft while minimizing tissue damage during insertion. For example, the minute vibrations of the shaft may create highly localized separation of subcutaneous tissue proximate the distal end of the shaft during insertion to create a highly precise subcutaneous thread path.

Subcutaneous Tunneling Tool

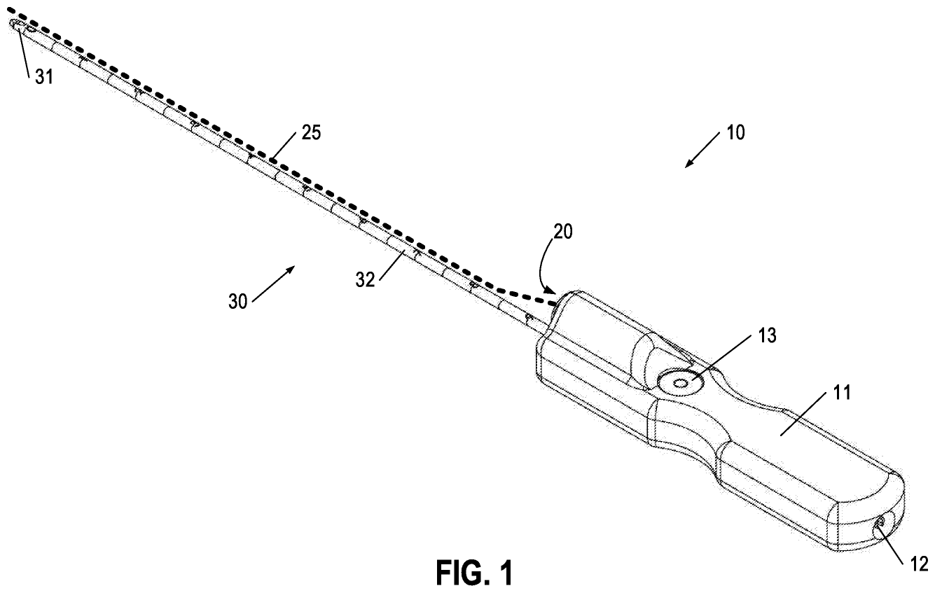

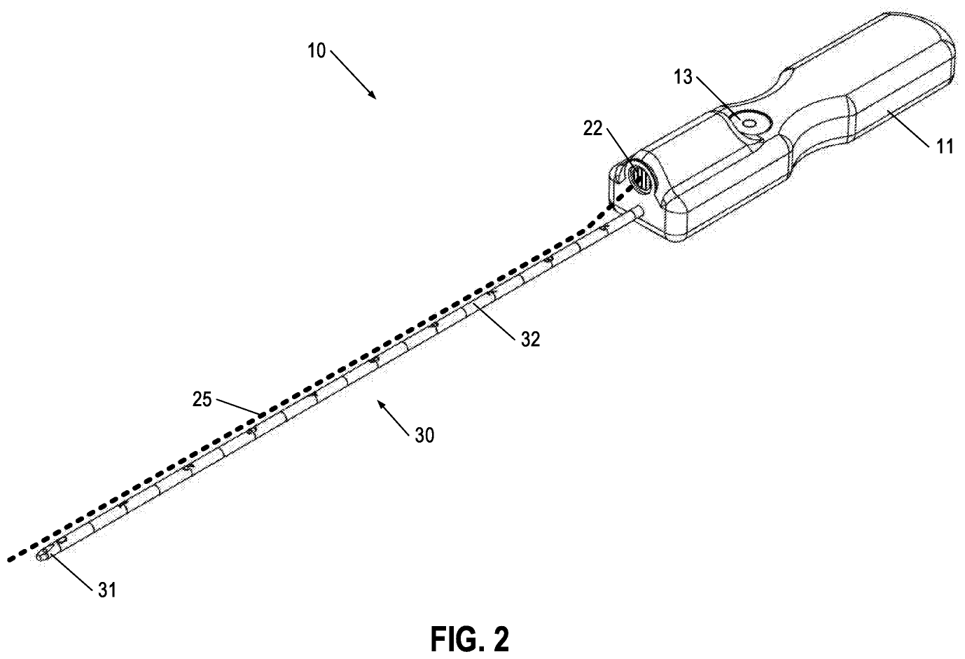

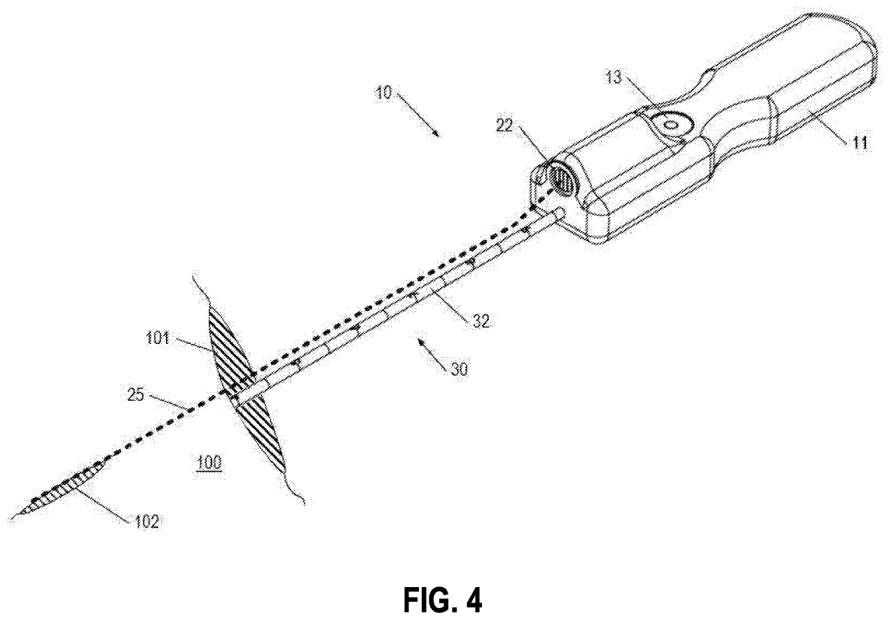

FIGS. 1-3 show various views of a subcutaneous tunneling tool 10 according to one embodiment. As shown in FIGS. 1-2, the subcutaneous tunneling tool 10 comprises a handle 11 having an elongated tunneling member 30 secured thereto. The handle 11 is configured to enable a user (e.g., a surgeon) to position and/or guide the elongated tunneling member 30 into a desired position, such as through an incision 101 in a patient's skin 100 (as shown in FIG. 4).

The handle 11 comprises a rigid material (e.g., a rigid plastic, metal, and/or the like). In certain embodiments, the handle 11 comprises one or more antimicrobial materials to ease sterilization of the handle 11 prior to use in a surgical setting. Although not shown, the handle 11 may comprise one or more grip portions (e.g., a resilient handle cover, one or more resilient portions within the handle exterior surface, and/or the like) to ease use of the subcutaneous tunneling tool 10.

The elongated tunneling member 30 may be rigidly secured relative to a first end of the handle 11. However, in certain embodiments at least a portion of the elongated tunneling member 30 may be detachably secured relative to the first end of the handle 11. In various embodiments the elongated tunneling member 30 may be secured relative to an aperture in the handle 11 with a friction fitting, however any of a variety of fastening mechanisms may be used (e.g., cotter pin, corresponding threading, interference fitting, and/or the like).

In the illustrated embodiment, the elongated tunneling member 30 comprises a tunneling shaft 31 having a proximal end secured to the handle 11 and an opposite distal end. In the illustrated embodiment of FIGS. 1-2, the tunneling shaft 31 is linear and cylindrical having an at least substantially uniform cross-sectional size and shape along the length of the elongated tunneling shaft 31 such that the tunneling shaft 31 is configured to create an at least substantially linear subcutaneous tunnel when inserted below a patient's skin. However, it should be understood that in certain embodiments, the tunneling shaft 31 may be flexible and/or may comprise one or more joints, or may be curved (e.g., to create a curved subcutaneous tunnel). Moreover, the tunneling shaft 31 may have any of a variety of cross-sectional shapes (e.g., ovular, square, rectangular, triangular, and/or the like). The tunneling shaft 31 may be a solid member comprising a metallic or otherwise conductive material (e.g., a surgical grade stainless steel). However, in certain embodiments the tunneling shaft 31 may be at least partially hollow.

Moreover, as shown in the figures, the distal end of the tunneling shaft 31 is chamfered to ease passage of the elongated tunneling member 30 through subcutaneous tissue when creating a subcutaneous tunnel. In certain embodiments, the distal end of the tunneling shaft 31 may be blunt, however in certain embodiments the chamfering may be configured to form a blade-like cutting tip at the distal end of the tunneling shaft 31. Moreover, the distal end of the tunneling shaft 31 may have a transverse through-hole therein, configured to enable a suture (or other medical-grade threading) to be secured to the tunneling shaft 31 such that the suture may be pulled through the subcutaneous tunnel formed by the tunneling member 30. The suture may be secured relative to the transverse through-hole prior to forming the subcutaneous tunnel (in which case the suture is pulled along with the distal end of the tunneling shaft 31 while the tunneling shaft forms the subcutaneous tunnel) or after forming the subcutaneous tunnel (in which case the suture may be pulled through the subcutaneous tunnel when the elongated tunneling member 30 is removed from the formed subcutaneous tunnel).

In the illustrated embodiment, the tunneling member 30 further comprises an insulating sleeve 32 surrounding the tunneling shaft 31. The insulating sleeve 32 is embodied as a hollow tubular member comprising an insulating material (e.g., a surgical grade plastic) and having opposite open ends and surrounding the tunneling shaft 31. A proximal end of the insulating sleeve 32 may be secured to the housing 11 and may be positioned concentric with the tunneling shaft 31. Moreover, as shown in the figures, the distal end of the tunneling shaft 31 extends beyond a distal end of the insulating sleeve 32, such that the distal end of the tunneling shaft 31 is exposed to subcutaneous tissue to enable the distal end of the tunneling shaft 31 to separate subcutaneous tissue while the tunneling member 30 is being inserted under a patient's skin. In embodiments in which the distal end of the tunneling shaft 31 is chamfered, the distal end of the insulating sleeve 32 may be aligned with the proximal edge of the chamfered portion of the tunneling shaft 31. As shown in the figures, the insulating sleeve 32 may have length demarcations provided (e.g., printed, etched, and/or the like) along the length of the insulating sleeve 32. As shown in the figures, the distal end of the insulating sleeve 32 may be indicated as a length datum, and various length demarcations may be provided extending away from the distal end of the insulation sleeve 32 (e.g., following repeating units, such as inches, centimeters, and/or the like) to provide an indication of the length of the tunneling member 30 inserted into a patient. In embodiments in which the distal end of the insulating sleeve 32 is aligned with a proximal end of chamfering on the tunneling shaft 31, the length demarcations may provide an indication of the length of a fully-formed (e.g., maximum diameter) subcutaneous tunnel.

The handle 11 houses one or more guide mechanisms, such as a leveling indicator (e.g., a bubble level 13), a beam emitter 20, and/or the like. In certain embodiments, the leveling indicator may be configured to aid users (e.g., surgeons) in maintaining a constant depth of insertion of the elongated tunneling member 30 within a patient. The leveling indicator may be rigidly secured relative to the handle 11, such that the leveling indicator is configured to indicate when the handle 11 (and therefore the entirety of the subcutaneous tunneling tool 10) is level. In certain embodiments, the leveling indicator is configured to determine whether the handle 11 is level about at least one axis of rotation (e.g., about an axis of rotation perpendicular to the length of the elongated tunneling member 30). For example, the leveling indicator may be configured to determine whether the handle 11 is level about two axes of rotation, including a first axis of rotation perpendicular to the length of the elongated tunneling member 30 and a second axis of rotation parallel with the length of the elongated tunneling member 30. In FIGS. 1-2, for example, the bubble level 13 is rigidly secured to the handle 11 and configured to indicate whether the handle is level about the first axis of rotation and the second axis of rotation (e.g., a bubble floating within a fluid within the bubble level 13 is aligned with a centrally located circle etched on the surface of the bubble level 13 when the handle 11 is level about both axes of rotation).

In certain embodiments, the leveling indicator is adjustably secured relative to the handle 11, such that the leveling indicator may be utilized to indicate a desired orientation of the handle 11. For example, the leveling indicator may be selectably and rigidly secured at a desired angle (about one or more axes of rotation) relative to the handle 11 to indicate whether the handle 11 is oriented at a desired angle. As a specific example, the handle 11 may be placed on a support surface having a desired angle relative to horizontal. While the handle 11 remains placed on the support surface, the leveling indicator may be adjusted to indicate that the support surface is level (e.g., the bubble level 13 may be adjusted such that the bubble is within the etched circle while the handle 11 is positioned on the support surface). Thereafter, the leveling indicator may be utilized to indicate whether the handle 11 is oriented parallel to the support surface, regardless of whether the support surface is level.

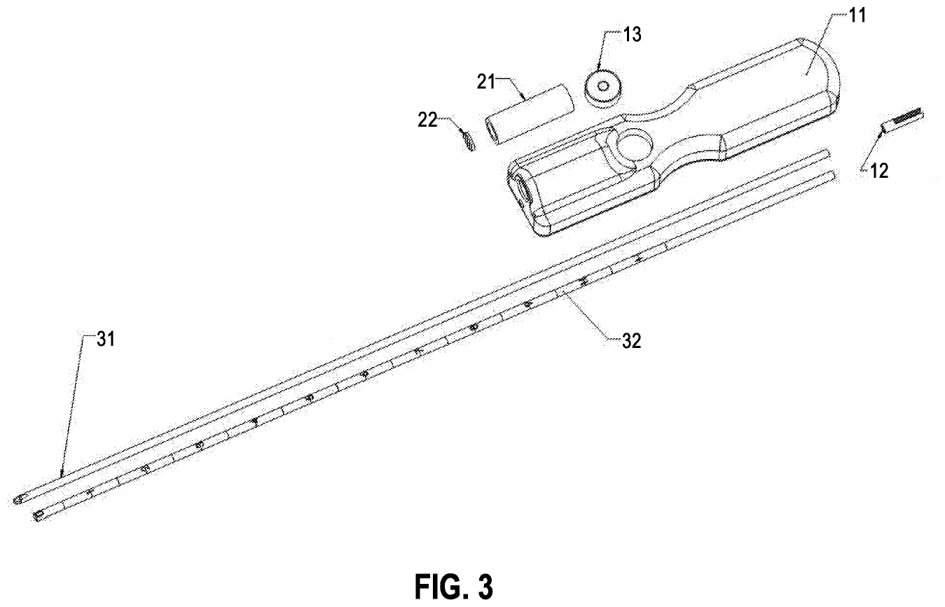

As mentioned, the handle 11 may further house a beam emitter 20 configured to emit a visible light beam that may be used to aid in positioning the elongated tunneling member 30 beneath a patient's skin. As shown in FIG. 3, the beam emitter 20 may comprise a light source (e.g., a laser emitter 21) and a lens 22 (e.g., a converging lens) configured to create a converged, visible light beam line that is aligned with the elongated tunneling member 30. For example, the beam emitter 20 may be configured to emit a laser line 25 as shown schematically in FIGS. 1-2 and 4.

As shown in FIG. 2, the beam emitter 20 is secured to and/or within the first end of the handle 11 and is spaced apart from the proximal end of the elongated tunneling member 30. The beam emitter 20 is configured to project a visible light beam (e.g., laser line 25) that is aligned with the elongated tunneling member 30 (e.g., aligned within a common plane). The emitted visible light beam is projected toward the elongated tunneling member 30 such that the visible light beam is projected onto an exterior surface of the elongated tunneling member 30 while the subcutaneous tunneling tool 10 is not in use, and the visible light beam is projected onto an exterior surface of a patient's skin when the elongated tunneling member 30 is positioned at least partially below the patient's skin. Accordingly, when the subcutaneous tunneling tool 10 is used to create a subcutaneous tunnel beneath the patient's skin, the user (e.g., surgeon) can guide the elongated tunneling member 30 based on the position of the visible light beam line. For example, the user may align the laser beam 25 of the illustrated embodiments with a desired subcutaneous tunnel path, and then insert the elongated tunneling member 30 through an incision in the patient's skin and along the subcutaneous tunnel path beneath the patient's skin, while the laser beam 25 remains aligned with the desired subcutaneous tunnel path. Moreover, in the illustrated embodiment, the laser beam 25 projects beyond the distal end of the elongated tunneling member 30, such that the elongated tunneling member 30 may be aligned with a desired subcutaneous tunnel path prior to forming the subcutaneous tunnel with the elongated tunneling member 30.

In the illustrated embodiment, both the elongated tunneling member 30 and the projected laser beam 25 are at least substantially linear. However, as mentioned above, the elongated tunneling member 30 may be curved in certain embodiments, and accordingly the projected laser beam 25 may likewise define a curved projected shape corresponding to the shape and positioning of the elongated tunneling member 30. In embodiments in which the elongated tunneling member 30 is flexible or otherwise adjustable, the beam emitter 20 may likewise be adjustable to project a laser line 25 that is aligned with the shape of the elongated tunneling member 30.

Moreover, in certain embodiments the subcutaneous tunneling tool 10 comprises a vibration generator configured to vibrate at least the tunneling shaft 31 of the elongated tunneling member 30 to ease insertion of the tunneling shaft 31 through subcutaneous tissue. The vibration generator may be configured to vibrate the tunneling shaft 31 at a high frequency (e.g., a radio frequency) to ease separation of the subcutaneous tissue encountered by the distal end of the tunneling shaft 31 during insertion. In certain embodiments, the concentric insulating sleeve 32 does not vibrate, and accordingly the distal end of the tunneling shaft 31 is the only vibrating portion of the elongated tunneling member 30 exposed to subcutaneous tissue surrounding the elongated tunneling member 30.

As shown in FIG. 1, the handle 11 may comprise an electrical power adapter 12 configured to receive power for at least the vibration generator and/or the beam emitter 20. Although not shown, the handle 11 may additionally or alternatively house one or more onboard power supplies, such as replaceable primary or rechargeable secondary batteries that may be used to provide electrical power to the vibration generator and/or the beam emitter 20.

Method of Creating a Subcutaneous Tunnel

The subcutaneous tunneling tool 10 is configured to create a subcutaneous tunnel below a patient's skin 100, for example to route conductors and/or sensor wires associated with ICD electrical leads and sensors between an ICD control module and a patient's heart.

FIG. 4 is a schematic illustration of a subcutaneous tunneling tool 10 utilized to create a subcutaneous tunnel within a patient. As shown in FIG. 4, the distal end of the elongated tunneling member 30 is inserted into a first incision 101 made within the skin 100 of the patient. During initial insertion, the laser beam 25 generated by the beam emitter 20 within the handle is aligned with a desired subcutaneous tunnel path (e.g., an at least substantially straight line between the first incision 101 and a second incision 102. For example, the subcutaneous tunneling tool 10 may be utilized to create a subcutaneous tunnel between a first incision 101 made proximate the patient's xyphiod process and a second, superior incision 102 made along the patient's sternum. It should be understood that the subcutaneous tunneling tool 10 may be utilized to create subcutaneous tunnels in other portions of the patient's body as well, such as between a location proximate the fifth intercostal space and the xiphoid process.

In use, while the distal end of the elongated tunneling member 30 is initially being inserted into the first incision 101 (defining the entry point of the subcutaneous tunnel), the laser beam 25 may be aligned with the second incision 102 (defining the end of the desired subcutaneous tunnel) as shown in FIG. 4 to align the elongated tunneling member 30 with a desired subcutaneous tunnel path between the first incision 101 and the second incision 102. As the elongated tunneling member 30 is inserted farther into the first incision 101 (thereby directing the distal end of the tunneling shaft 31 to separate subcutaneous tissues along the desired subcutaneous tunnel path) the laser beam 25 remains aligned with the second incision 102 to guide the elongated tunneling member 30 along the desired subcutaneous tunnel path.

Moreover, the leveling indicator may be utilized to aid in maintaining a desired subcutaneous tunnel depth as the elongated tunneling member 30 is inserted into the first incision. For example, a bubble level 13 may be configured such that the included bubble remains within an etched central circle within the bubble level 13 when inserting the elongated tunneling member 30 at a constant depth.

In certain embodiments, the subcutaneous tunneling tool 10 comprises a vibration mechanism configured to vibrate the tunneling shaft 31 at a predefined frequency (e.g., at a radio frequency) to facilitate separating subcutaneous tissue encountered by the distal end of the tunneling shaft 31 while the elongated tunneling member 30 is inserted into the patient's body. As mentioned, the elongated tunneling member 30 may comprise an insulating sleeve 32 surrounding the tunneling shaft 31 configured to insulate the patient's body from the vibrating tunneling shaft 31. As shown in FIGS. 1-2, only the distal end of the tunneling shaft 31 may be exposed relative to the insulating sleeve 32, such that the vibration may be utilized to separate subcutaneous tissue encountered by the distal end of the tunneling shaft 31.

The subcutaneous tunneling tool 10 may be further configured to aid in threading sutures, electrical leads, sensors, and/or the like through subcutaneous tunnels. As mentioned a transverse through-hole may be disposed through the tunneling shaft 31 proximate the distal end thereof. A suture, thread, or other apparatus may be secured relative to the transverse through-hole, and may be pulled through the subcutaneous tunnel by the tunneling shaft 31 during and/or after formation of the subcutaneous tunnel. Once the suture is threaded through the subcutaneous tunnel and the elongated tunneling member 30 is removed from the formed subcutaneous tunnel, an electrical lead, sensor, or other device may be secured relative to an end of the suture, and pulled through the created subcutaneous tunnel.

### CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A subcutaneous tunneling tool comprising:
an elongated shaft configured to be inserted below a patient's skin to create a subcutaneous tunnel; and
a housing comprising a guide mechanism secured to an end of the elongated shaft, wherein the guide mechanism is configured to project a visible light beam aligned with the elongated shaft toward the elongated shaft.

2. The subcutaneous tunneling tool of claim 1, wherein the guide mechanism comprises:
a beam generator secured to the housing and spaced apart from the elongated shaft, wherein the beam generator is configured to project the visible light beam toward the elongated shaft.

3. The subcutaneous tunneling tool of claim 2, wherein the guide mechanism further comprises a leveling indicator secured to the housing.

4. The subcutaneous tunneling tool of claim 2, wherein the beam generator is configured to project a laser line toward the elongated shaft, wherein the laser line is aligned with the elongated shaft.

5. The subcutaneous tunneling tool of claim 4, wherein the laser line is longer than the elongated shaft.

6. The subcutaneous tunneling tool of claim 1, wherein the elongated shaft comprises a metallic material.

7. The subcutaneous tunneling tool of claim 5, further comprising an elongated sheath surrounding the elongated shaft.

8. The subcutaneous tunneling tool of claim 5, wherein the elongated sheath has length demarcations thereon.

9. The subcutaneous tunneling tool of claim 1, further comprising a vibration emitter configured to vibrate the elongated shaft at a defined frequency to separate subcutaneous tissue of the patient when the elongated shaft is inserted below the patient's skin.

10. The subcutaneous tunneling tool of claim 8, wherein the defined frequency is a radio frequency.

11. A method for creating a subcutaneous tunnel in a patient, the method comprising:
inserting a distal end of an elongated shaft through an incision in the patient's skin;
projecting a guide beam onto an exterior surface the patient's skin, wherein the guide beam is projected from a guide mechanism secured to a proximal end of the elongated shaft and the guide beam is aligned with the elongated shaft;
aligning the guide beam with a desired subcutaneous tunnel path; and
inserting the elongated shaft through the desired subcutaneous tunnel path.

12. The method of claim 11, further comprising vibrating the elongated shaft at a desired frequency to separate subcutaneous tissue along the desired subcutaneous tunnel path.

13. The method of claim 12, wherein the desired frequency is a radio frequency.

14. The method of claim 11, further comprising steps for aligning a leveling indicator within the guide mechanism with a level configuration and maintaining the leveling indicator in the level configuration while inserting the elongated shaft through the desired subcutaneous tunnel path.

15. The method of claim 11, wherein the elongated shaft is surrounded by a sheath having length demarcations thereon, and wherein inserting the elongated shaft through the desired subcutaneous tunnel path comprises inserting the elongated shaft through the incision to a desired length demarcation.

16. The method of claim 15, wherein the distal end of the elongated shaft is exposed beyond a distal end of the sheath, wherein the method further comprises vibrating the elongated shaft at a desired frequency within the sheath to separate subcutaneous tissue at the distal end of the elongated shaft.
